(19) United States
(12) Patent Application Publication
(10) Pub. No.: US 2002/0137152 A1
(43) Pub. Date: Sep. 26, 2002

(54) Fermentation process for epothilones

(76) Inventors: Daniel Santi, San Francisco, CA (US); Brian Metcalf, Moraga, CA (US); Gary Ashley, Alameda, CA (US)

Correspondence Address: Carolyn A. Favorito, Morrison & Foerster LLP, Suite 500, 3811 Valley Centre Drive, San Diego, CA, 92130-2332, US

(21) Appl. No.: 09/916,045

(22) Filed: Jul. 25, 2001

Related U.S. Application Data
(60) Provisional application No. 60/220,651, filed on Jul. 25, 2000.

(51) Int. Cl. C12P 17/16; C12N 9/99; C12N 1/21

(52) U.S. Cl. 435/118; 435/184; 435/252.3

(57) ABSTRACT

Desoxyepothilone compounds are produced by fermentation of an epothilone producing microorganism in the presence of a P450 enzyme inhibitor.

### CROSS-REFERENCE TO RELATED APPLICATIONS

This patent application claims priority under 35 U.S.C. § 119(e) to U.S. provisional application Ser. No. 60/220,651, filed Jul. 25, 2000, which is incorporated herein by reference.

### FIELD OF THE INVENTION

The present invention provides methods and materials for producing epothilone and epothilone derivatives. The invention relates to the fields of agriculture, chemistry, medicinal chemistry, medicine, molecular biology, and pharmacology.

### BACKGROUND OF THE INVENTION

The epothilones were first identified by Gerhard Hofle and colleagues at the National Biotechnology Research Institute as an antifungal activity extracted from the myxobacterium Sorangium cellulosum (see K. Gerth et al., 1996, J. Antibiotics 49:560-563 and Germany Patent No. DE 41 38 042). The epothilones were later found to have activity in a tubulin polymerization assay (see D. Bollag et al., 1995, Cancer Res. 55:2325-2333) to identify antitumor agents and have since been extensively studied as potential antitumor agents for the treatment of cancer.

The chemical structure of the epothilones produced by Sorangium cellulosum strain So ce 90 was described in Hofle et al., 1996, “Epothilone A and B—novel 16-membered macrolides with cytotoxic activity: isolation, crystal structure, and conformation in solution,” Angew. Chem. Int. Ed. Engl. 35(13/14): 1567-1569, incorporated herein by reference. The strain was found to produce two epothilone compounds, designated A (R═H) and B (R═CH₃), as shown below, which showed broad cytotoxic activity against eukaryotic cells and noticeable activity and selectivity against breast and colon tumor cell lines.

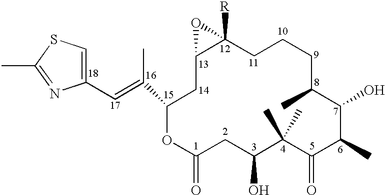

The desoxy counterparts of epothilones A and B, also known as epothilones C (R═H) and D (R═CH₃), are known to be less cytotoxic, and the structures of these epothilones are shown below.

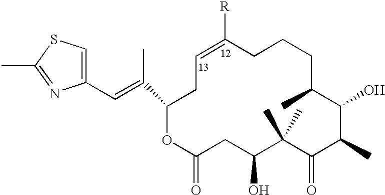

Other naturally occurring epothilones have been described. These include epothilones E and F, in which the methyl side chain of the thiazole moiety of epothilones A and B has been hydroxylated to yield epothilones E and F, respectively.

Because of the potential for use of the epothilones as anticancer agents, and because of the low levels of epothilone produced by the native So ce 90 strain, a number of research teams undertook the effort to synthesize the epothilones. This effort has been successful (see Balog et al., 1996, Total synthesis of (−)-epothilone A, Angeic. Chem. Int. Ed. Engl. 35(23/24):2801-2803; Su et al., 1997, “Total synthesis of (−)-epothilone B: an extension of the Suzuki coupling method and insights into structure-activity relationships of the epothilones,” Angew. Chem. Int. Ed. Engl. 36(7):757-759; Meng et al., 1997, “Total syntheses of epothilones A and B,” JACS 119(42):10073-10092; and Balog et al., 1998, “A novel aldol condensation with 2-methyl-4-pentenal and its application to an improved total synthesis of epothilone B,” Angew. Chem. Int. Ed. Engl. 37(19):2675-2678, each of which is incorporated herein by reference). Despite the success of these efforts, the chemical synthesis of the epothilones is tedious, time-consuming, and expensive. Indeed, the methods have been characterized as impractical for the full-scale pharmaceutical development of an epothilone.

A number of epothilone derivatives, as well as epothilones A-D, have been studied in vitro and in vivo (see Su et al., 1997, “Structure-activity relationships of the epothilones and the first in vivo comparison with paclitaxel,” Angew. Chem. Int. Ed. Engl. 36(19):2093-2096; and Chou et al., August 1998, “Desoxyepothilone B: an efficacious microtubule-targeted antitumor agent with a promising in vivo profile relative to epothilone B,” Proc. Natl. Acad. Sci. USA 95:9642-9647, each of which is incorporated herein by reference). Additional epothilone derivatives and methods for synthesizing epothilones and epothilone derivatives are described in PCT patent publication Nos. 99/54330, 99/54319, 99/54318, 99/43653, 99/43320, 99/42602, 99/40047, 99/27890, 99/07692, 99/02514, 99/01124, 98/25929, 98/22461, 98/08849, and 97/19086; U.S. Pat. No. 5,969,145; and Germany patent publication No. DE 41 38 042, each of which is incorporated herein by reference.

Of the naturally occurring epothilones studied to date, epothilone D appears to have the lowest toxicity (see Chou et al., 1998, Proc. Nat. Acad. Sci. 95:15798 and Chou et al., 1998, Proc. Nat. Acad. Sci. 95: 9642) and greatest efficacy (see Harris et al., 1999, Soc. Chim. Ther. 25:187). However, epothilone D is produced in very low amounts in the Sorangium cellulosum host cells that naturally produce the compound. Moreover, epothilone D is produced in those cells as a minor component in a complex mixture of epothilones.

There remains a need for economical means to produce epothilone D and other desoxyepothilones in amounts needed for clinical trials and, if those trials are successful, human therapeutic use. If sufficient quantitities of epothilone D were available, then new epothilone D derivatives with improved properties could be produced. The present invention meets these and other needs.

### BRIEF DESCRIPTION OF THE DRAWINGS

FIG. 1 shows the production of epothilones A, B, C, and D by the Sorangium cellulosum strain So ce 90 in the presence of various P450 inhibitors.

FIG. 2 shows the effect of metyrapone on the growth of the Sorangium cellulosum strain So ce 90.

### SUMMARY OF THE INVENTION

In one embodiment, the present invention provides a process for producing a desoxyepothilone, an epothilone lacking the C-12 to C-13 epoxide moiety found in epothilones A and B, by fermentation of an epothilone producing microorganism in the presence of an inhibitor of an epothilone epoxidase gene product, such as EpoK. In one aspect, the microorganism is Sorangium cellulosum. In another aspect, the microorganism is a recombinant microorganism that contains the epothilone biosynthetic gene cluster.

In another embodiment, the present invention provides inhibitors of the epothilone epoxidase gene product EpoK and methods for making such compounds.

In another embodiment, the present invention provides a recombinant Sorangium cellulosum in which the epoK gene has been inactivated by random mutagenesis and so produces only epothilones C and D. In another embodiment, this recombinant microorganism produces only epothilone C or epothilone D due to an alteration in the genes coding for the epothilone polyketide synthase (PKS).

These and other embodiments of the invention are described in more detail in the following description, the examples, and claims set forth below.

### DETAILED DESCRIPTION OF THE INVENTION

The present invention provides methods and reagents useful in the production of the desoxyepothilones, particularly epothilones C and D. The epothilones (epothilone A, B, C, D, E, and F) and compounds structurally related thereto (epothilone derivatives) are potent cytotoxic agents specific for eukaryotic cells. These compounds have application as anti-fungals, cancer chemotherapeutics, and immunosuppressants. The epothilones are produced at very low levels in the naturally occurring Sorangium cellulosum cells in which they have been identified.

Sorangium cellulosum produces a number of structurally related epothilones. Epothilones A and B are most abundantly produced and were the first epothilones discovered (see PCT patent publication No. 93/10121, incorporated herein by reference). Epothilones A and B contain an epoxide moiety at C-12 to C-13 and differ in this regard from their corresponding analogs, epothilones C and D, which contain a C-C double bond at this position. Epothilones C and D are produced in much lower amounts in Sorangium cellulosum (see PCT patent publication No. 97/19086, incorporated herein by reference) than epothilones A and B. Further analysis has shown that the producer organism produces a relatively large number of different epothilone analogs (see PCT patent publication Nos. 98/22461 and 99/65913, each of which is incorporated herein by reference).

The mechanisms by which the epothilones are produced have been determined in part by the cloning and characterization of the genes encoding the enzyme activities in the epothilone biosynthetic pathway (see PCT patent publication Nos. 00/031247; see also PCT patent application No. 99/66028, each of which incorporated herein by reference). U.S. patent application Ser. No. 09/443,501, filed Nov. 19, 1999, incorporated herein by reference, discloses the nucleotide sequence of the epothilone biosynthetic gene cluster for a linear segment of ˜72 kb of Sorangium cellulosum chromosomal DNA. Analysis revealed a polyketide synthase (PKS) gene cluster with a loading domain and nine modules. Downstream of the PKS sequences is an ORF, designated epoK, that shows strong homology to cytochrome P450 oxidase genes and encodes the epothilone epoxidase.

The epothilone PKS genes are organized in 6 open reading frames. At the polypeptide level, the loading domain and modules 1, 2, and 9 appear on individual polypeptides; their corresponding genes are designated epoA, epoB, epoC, and epoF respectively. Modules 3, 4, 5, and 6 are contained on a single polypetide whose gene is designated epoD, and modules 7 and 8 are on another polypeptide whose gene is designated epoE . It is clear from the spacing between ORFs that epoC, epoD, epoE and epoF constitute an operon. The epoA, epoB, and epok gene may be also part of the large operon, but there are spaces of approximately 100 bp between epoB and epoC and 115 bp between epoF and epoK which could contain a promoter. The epothilone biosynthetic gene cluster is shown schematically below.

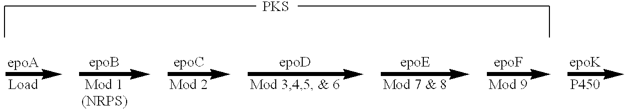

A detailed examination of the modules shows an organization and composition that is consistent with one able to be used for the biosynthesis of epothilone. The description that follows is at the polypetide level. The sequence of the acyltransferase (AT) domain in the loading module and in modules 3, 4, 5, and 9 shows similarity to the consensus sequence for malonyl specifying AT domains, consistent with the presence of an H side chain at C-14, C-12 (epothilones A and C), C-10, and C-2, respectively, as well as the loading domain. The AT domains in modules 2, 6, 7, and 8 resemble the consensus sequence for methylmalonyl specifying AT domains, again consistent with the presence of methyl side chains at C-16, C-8, C-6, and C-4 respectively.

The loading module contains a ketosynthase (KS) domain in which the cysteine residue usually present at the active site is instead a tyrosine. This domain is designated as KS^{y} and serves as a decarboxylase, which is part of its normal function, but cannot function as a condensing enzyme. Thus, the loading domain is expected to load malonyl CoA, move it to the acyl carrier protein (ACP), and decarboxylate it to yield the acetyl residue required for condensation with cysteine.

Module 1 is a non-ribosomal peptide synthetase (NRPS) that activates cysteine and catalyzes the condensation with acetate on the loading module. The sequence contains segments highly similar to ATP-binding and ATPase domains required for activation of amino acids, a phosphopantotheinylation site, and an elongation domain.

Module 2 determines the structure of epothilone at C-15 -C-17. The presence of the dehydratase (DH) domain in module 2 yields the C-16 - C-17 dehydro moiety in the molecule. The domains in module 3 are consistent with the structure of epothilone at C-14 and C-15; the OH that comes from the action of the ketoreductase (KR) is employed in the lactonization of the molecule.

Module 4 controls the structure at C-12 and C-13, where a double bond is found in epothilones C and D, consistent with the presence of a DH domain. Although the sequence of the AT domain appears to resemble those that specify malonate loading, it can also load methylmalonate, thereby accounting in part for the mixture of epothilones found in the fermentation broths of the naturally producing organisms. A significant departure from the expected array of functions was found in module 4. This module was expected to contain a DH domain, thereby directing the synthesis of epothilones C and D as the products of the PKS. Rigorous analysis revealed that the space between the AT and KR domains of module 4 was not large enough to accommodate a functional DH domain. Thus, the extent of reduction at module 4 does not proceed beyond the ketoreduction of the beta-keto formed after the condensation directed by module 4. Because the C-12, 13 unsaturation has been demonstrated (epothilones C and D), there must be an additional dehydratase function that introduces the double bond, and this function is believed to be in the PKS itself, as epothilones C and D are produced in heterologous host cells comprising the epothilone PKS genes.

Modules 5 and 6 each have the full set of reduction domains (KR, DH and enoylreductase (ER)) to yield the methylene functions at C-11 and C-9. Modules 7 and 9 have KR domains to yield the hydroxyls at C-7 and C-3, and module 8 does not have a functional KR domain, consistent with the presence of the keto group at C-5. Module 8 also contains a methyltransferase (MT) domain that results in the presence of the geminal dimethyl function at C-4. Module 9 has a thioesterase domain that terminates polyketide synthesis and catalyzes ring closure. The genes, proteins, modules, and domains of the epothilone PKS are summarized in the following Table.

| Gene | Protein | Modules | Domains Present |
|---|---|---|---|
| epoA | EpoA | Load | Ks^{y} mAT ER ACP |
| epoB | EpoB | 1 | NRPS, condensation, heterocyclization, |
| | | | adenylation, thiolation, PCP |
| epoC | EpoC | 2 | KS mmAT DH KR ACP |
| epoD | EpoD | 3-6 | KS mAT KR ACP; KS mAT KR ACP; KS |
| | | | mAT DH ER KR ACP; KS mmAT DH |
| | | | ER KR ACP |
| epoE | EpoE | 7-8 | KS mmAT KR ACP; KS mmAT MT DH* |
| ACP | | | KR* |
| epoF | EpoF | 9 | KS mAT KR DH* ACP TE |

NRPS-non-ribosomal peptide synthetase; KS-ketosynthase; mAT-malonyl CoA specifying acyltransferase; mmAT-methylmalonyl CoA specifying acyltransferase; DH-dehydratase; ER-enoylreductase; KR-ketoreductase; MT-methyltransferase; TE thioesterase;*-inactive domain.

From an analysis of the cloned genes in the epothilone biosynthetic gene cluster and from heterologous production of epothilones, the biosynthetic pathway was deduced to be as follows. First, the epothilone PKS produces epothilones C and D, depending on whether the AT domain of module 4 binds malonyl CoA (to form epothilone C) or methylmalonyl CoA (to form epothilone D). Then, the epoK gene product acts on epothilones C and D to form the epoxidated derivatives epothilones A and B, respectively.

Despite being first in the order of synthesis in Sorangium cellulosum, epothilones C and D are produced in much lower abundance than epothilones A and B in all natural isolates reported to date. Because the non-epoxidated epothilones C and D are less toxic than their epoxidated counterparts, the lack of an efficient fermentation process for their production is a significant barrier to the development of improved cancer therapies. The present invention provides a means of overcoming this barrier to produce epothilones C and D in abundance.

In one embodiment, the present invention provides a method for preparing epothilones C and D in greater abundance than they are produced in Sorangium cellulosum So ce 90 (DSM 6773). In one mode, epothilones C and D are produced in greater abundance than epothilones A and B. In another mode, only epothilones C and D are produced. In another mode, only epothilone C is produced. In another mode, only epothilone D is produced.

In another embodiment, the present inventon provides a method for preparing epothilones C and D by fermentation of a Sorangium cellulosum host cell in the presence of an inhibitor of a P450 enzyme. In one mode, the inhibitor is a reversible inhibitor. In another mode, the inhibitor is an irreversible inhibitor. In a preferred mode, the inhibitor is a specific inhibitor of the epoK gene product. In one embodiment, the inhibitor is metyrapone (2-methyl-1,2-di-3-pyridyl-1-propanone).

Inhibitors of the epoK gene product can be readily identified using an in vitro assay using a recombinant EpoK enzyme and a panel of putative inhibitors. The production of the recombinant EpoK enzyme and the in vitro assay are described in Example 1, below. There are numerous known P450 enzyme inhibitors that can be tested in this assay and, if effective, employed in the methods of the present invention. Such P450 inhibitors include, but are not limited to, ketoconazole, itraconazole, miconazole, furafylline, sulfaphenazole, proadifen, debrisoquin, and derivatives thereof. In a preferred embodiment, the inhibitor is a member of the class of acetylenic mechanism-based irreversible inhibitors.

In one embodiment, the present invention provides specific and irreversible inhibitors of EpoK. These inhibitors are represented by the generic structure:

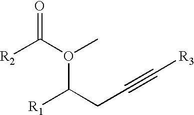

wherein R₁ is aryl, heterocycle, aryl—CH═CR₄—, or heterocycle—CH═CR₄; R₂ is lower alkyl (C1-C6) or substituted alkyl preferably C₁₋₃ alkyl; R₃ is H or is lower alkyl (C1-C6) or substituted alklyl, preferably methyl, or ethyl; and R₄ is H or is lower alkyl (C1-C6) or substituted alkyl, preferably methyl.

The term aryl as used herein refers to a mono or bicyclic carbocyclic ring system having one or two aromatic rings including but not limited to phenyl, naphthyl, tetrahydronaphthyl, indanyl, indenyl, and the like. Aryl groups, including bicyclic aryl groups, can be unsubstituted or substituted with one, two, or three substituents independently selected from lower alkyl (C1-C6), substituted lower alkyl, haloalkyl, alkoxy, thioalkoxy, amino, alkylamino, dialkylamino, acylamino, cyano, hydroxy, halo, mercapto, nitro, carboxaldehyde, carboxy, alkoxycarbonyl, and carboxamide. In addition, substituted aryl groups include tetraflurophenyl and pentaflurophenyl.

As used herein, a substituent that comprises an aromatic moiety contains at least one aromatic ring, such as phenyl, pyridyl, pyrimidyl, thiophenyl, or thiazolyl. The substituent may also include fused aromatic residues such as naphthyl, indolyl, benzothiazolyl, and the like. The aromatic moiety may also be fused to a nonaromatic ring and/or may be coupled to the remainder of the compound in which it is a substituent through a nonaromatic, for example, alkylene residue. The aromatic moiety may be substituted or unsubstituted as may the remainder of the substituent.

As used herein, the term lower alkyl refers to a C₁-C₃ alkyl, C₁-C₆ alkyl, and C₁-C₁₂ alkyl saturated, straight or branched chain hydrocarbon radicals derived from a hydrocarbon moiety containing between one and three, one and six, and one and twelve carbon atoms, respectively by removal of a single hydrogen atom. Examples include but are not limited to methyl, ethyl, propyl, isopropyl, n-butyl, tert-butyl, neopentyl, n-hexyl, n-heptyl, n-octyl, n-nonyl, n-decyl, n-undecyl, and n-dodecyl.

The term alkoxy refers to a lower alkyl group attached to a parent moiety through an oxygen atom. Examples include but are not limited to methoxy, ethoxy, propoxy, isopropoxy, n-butoxy, tert-butoxy, neopentoxy, and n-hexoxy.

The terms halo and halogen as used herein refer to an atom selected from fluorine, chlorine, bromine, and iodine. The term haloalkyl as used herein denotes a lower alkyl group to which one, two, or three halogen atomes are attached to any one carbon and includes without limitation chloromethyl, bromoethyl, trifluoromethyl, and the like.

The term heteroaryl as used herein refers to a cyclic aromatic radical having from five to ten ring atoms of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isoxazolyl, thiadiazolyl, oxadiazolyl, thiophenyl, furanyl, quinolinyl, isoquinolinyl, and the like.

The term heterocyle includes but is not limited to pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term substituted as used herein refers to a group substituted by independent replacement of one, two, or three of the hydrogen atoms thereon with Cl, Br, F, I, OH, CN, lower alkyl, lower alkoxy, lower aLkoxy substituted with aryl, haloalkyl, thioalkoxy, amino, alkylamino, dialkylamino, mercapto, nitro, carboxaldehyde, carboxy, alkoxycarbonyl, and carboxamide. Any one substituent may be an aryl, heteroaryl, or heterocycloalkyl group.

In a preferred embodiment, the inhibitor is selected from the group consisting of metyrapone, 1-phenyl-3-butyn-1-yl-acetate, 1-phenylhexen-5-yn-3-yl acetate, 1-(3-pyridyl)-3-butyn-1-yl acetate 1-(3-pyridyl)hexen-5-yn-3-yl acetate, 1-(4-pyridyl)-3-butyn-1-yl acetate, and 1-(4-pyridyl)hexen-5-yn-3-yl acetate. Methods for making these compounds are described in Example 2, below. Thus, in one embodiment, the present invention provides a method for producing the desoxy epothilones by fermentation of Sorangium cellulosum in the presence of a P450 enzyme inhibitor selected from the group consisting of 1-phenyl-3-butyn-1-yl-acetate, 1-phenylhexen-5-yn-3-yl acetate, 1-(3-pyridyl)-3-butyn-1-yl acetate 1-(3-pyridyl)hexen-5-yn-3-yl acetate, 1-(4-pyridyl)-3-butyn-1-yl acetate, and 1-(4-pyridyl)hexen-5-yn-3-yl acetate.

Example 3 describes a fermentation protocol for producing epothilones in Sorangium cellulosum. Generally, a culture medium for the preparation of epothilones contains the microorganism that produces these compounds, typically a myxobacteria such as Sorangium cellulosum So ce 90 (see PCT publication 93/10121) or a modified form thereof, in a medium that contains water and other conventional and appropriate constituents of culture media, such as biopolymers, sugar, amino acids, salts, nucleic acids, vitamins, antibiotics, growth media, extracts from biomaterials such as yeast or other cell extracts, soy meal, starch, such as potato starch, and or trace elements, for example iron ions in complex bound form, or suitable combinations of all or some of these constituents. Suitable culture media are known to the person skilled in the art or may be produced by known processes (see e.g. the culture media in the exmaples of PCT publication 93/10121). As noted, a preferred Sorangium is strain So ce 90 has been deposited under accession number DSM 6773 at the German Collection of Microorganisms and Cell Cultures (DSMZ, Braunschweig, Germany). Example 4 describes a protocol for producing the epothilines C and D in Sorangium cellulosum by inhibiting epoK with metyrapone. Metyrapone is included in the production medium at an effective effective to inhibit EpoK and increase production of epothilones C and D, but not detrimentally inhibiting the growth of Sorangium cellulosum.

In accordance with the methods of the invention, an epothilone producing Sorangium strain is fermented in a media containing a reversible or irreversible inhibitor of EpoK. Using this method, one can produce more of the desoxyepothilones than would be produced using the identical strain and methodology in the absence of the inhibitor. Depending on the amount and nature of inhibitor employed in the method, one can completely suppress formation of the epoxidated epothilones. However, because certain inhibitors and certain concentrations of inhibitors may be deleterious to cell growth, one may choose to practice the invention such that EpoK is not completely inhibited, and some production of the epoxidated epothilones is observed. Those of skill in the art will appreciate that, while Sorangium is a preferred epothilone producer for purposes of the present invention, the methods and compounds of the invention are useful with any epothilone producing cell or system in which EpoK or another epothilone epoxidating enzyme is present.

The inhibitors of the invention are added to the fermentation media to achieve a final concentration in the media in the range of 1 nM to 1 M, preferably in the range of 1 μM to 100 mM, most preferably in the range of 10 μM to 10 mM. The inhibitor can be added to the fermentation as a bolus or in aliquots over time; typically, the inhibitor will be added prior to the start of production of the epothilones.

In an alternative embodiment, the present invention provides a Sorangium host cell in which the epoK gene has been inactivated by mutation. The resulting mutant strain produces a greatly reduced or no amount of epothilones A and B, as compared to the unmutated strain, due to the absence of functional EpoK. Such mutant strains can be obtained by one or more mutagenesis steps, such as for example, UV-induced mutagenesis by radiation in the range of 200 to 400 nm, more particularly 250 to 300 nm, followed by identification of those mutants that produce lower amounts, relative to unmutated counterpart strains, of epothilones A and B and increased amounts of epothilones C and D. Example 5 describes the methodology for obtaining such mutant strains.

Thus, the invention provides two different methods for making the desoxyepothilones. In the first, an inhibitor of EpoK is added to the fermentation media, and in the second, a mutant Sorangium strain containing a mutationally inactivated epoK gene is fermented. Both methods can be practiced with strains that have been further modified to produce epothilone derivatives of interest. In many instances, these further modifications alter the epothilone PKS genes and the enzymatic function of the PKS.

Homologous recombination can be used to delete, disrupt, or alter a gene. The process of homologous recombination employs a vector that contains DNA homologous to the regions flanking the gene segment to be altered and positioned so that the desired homologous double crossover recombination event desired will occur. U.S. Pat. No. 5,686,295, incorporated herein by reference, describes a method for transforming Sorangium host cells by homologous recombination, although other methods may also be employed. Thus, homologous recombination can be used to alter the specificity of a PKS module by replacing coding sequences for the module or domain of a module to be altered with those specifying a module or domain of the desired specificity.

In one preferred embodiment, the present invention is practiced using a recombinant epothilone producing Sorangium cellulosum host cell in which the coding sequence for the AT domain of module 4 encoded by the epoD gene has been altered by homologous recombination to encode an AT domain that binds only methylmalonyl CoA. This host cell, fermented in accordance with the methods of the invention, is a preferred source of epothilone D. In another embodiment, the present invention is practiced using a recombinant epothilone producing Sorangium cellulosum host cell in which the coding sequence for the AT domain of module 4 encoded by the epoD gene has been altered by homologous recombination to encode an AT domain that binds only malonyl CoA. This host cell, fermented in accordance with the methods of the invention, is a preferred source of epothilone C.

In other embodiments, the Sorangium host cell comprises epothilone PKS genes that contain alterations other than or in addition to the alteration of the module 4 AT domain coding sequences to make other preferred epothilone derivatives. Such alterations include those that result in epothilone PKS enzymes that contain, relative to the native PKS, inserted KR, DH, or ER domains, deleted KR, DH, or ER domains, and replacement AT domains. Epothilone analogs that can be produced using this methodology include the 14-methyl epothilone derivatives (made by utilization of a hybrid module 3 that has an AT that binds methylmalonyl CoA instead of malonyl CoA); the 8,9-dehydro epothilone derivatives (made by utilization of a hybrid module 6 that has a DH and KR instead of an ER, DH, and KR); the 10-methyl epothilone derivatives (made by utilization of a hybrid module 5 that has an AT that binds methylmalonyl CoA instead of malonyl CoA); the 9-hydroxy epothilone derivatives (made by utilization of a hybrid module 6 that has a KR instead of an ER, DH, and KR); the 8-desmethyl-14methyl epothilone derivatives (made by utilization of a hybrid module 3 that has an AT that binds methylmalonyl CoA instead of malonyl CoA and a hybrid module 6 that binds malonyl CoA instead of methylmalonyl CoA); and the 8-desmethyl-8,9-dehydro epothilone derivatives (made by utilization of a hybrid module 6 that has a DH and KR instead of an ER, DH, and KR and an AT that specifies malonyl CoA instead of methylmalonyl CoA).

Those of skill in the art will thus appreciate that the methods of the present invention can be used to produce a wide variety of desoxyepothilones. These production methods are superior to those known in the art, because the desoxyepothilones are produced in less complex mixtures, containing lesser amounts or none of the epoxidated epothilones. In many embodiments, only a single desired desoxy epothilone compound is produced. Such host cells include those that make only epothilone D and those that make only epothilone C.

The host cells of the invention can be grown and fermented under conditions known in the art for other purposes to produce the compounds of the invention. The compounds of the invention can be isolated from the fermentation broths of these cultured cells and purified by standard procedures. Fermentation conditions for producing the compounds of the invention from Sorangium host cells can be based on the protocols described in PCT patent publication Nos. 93/10121, 97/19086, 98/22461, and 99/42602, each of which is incorporated herein by reference. The epothilones produced using the methods of the invention can be derivatized and formulated as described in PCT patent publication Nos. 93/10121, 97/19086, 98/08849, 98/22461, 98/25929, 99/01124, 99/02514, 99/07692, 99/27890, 99/39694, 99/40047,99/42602, 99/43653, 99/43320, 99/54319, 99/54319, and 99/54330, and U.S. Pat. No. 5,969,145, each of which is incorporated herein by reference.

Compounds produced in accordance with the methods of the invention can be readily formulated to provide pharmaceutical compositions. The pharmaceutical compositions can be used in the form of a pharmaceutical preparation, for example, in solid, semisolid, or liquid form. This preparation will contain one or more of the compounds of the invention as an active ingredient in admixture with an organic or inorganic carrier or excipient suitable for external, enteral, or parenteral application. The active ingredient may be compounded, for example, with the usual non-toxic, pharmaceutically acceptable carriers for tablets, pellets, capsules, suppositories, pessaries, solutions, emulsions, suspensions, and any other form suitable for use.

The carriers which can be used include water, glucose, lactose, gum acacia, gelatin, mannitol, starch paste, magnesium trisilicate, talc, corn starch, keratin, colloidal silica, potato starch, urea, and other carriers suitable for use in manufacturing preparations, in solid, semi-solid, or liquified form. In addition, auxiliary stabilizing, thickening, and coloring agents and perfumes may be used. For example, the compounds of the invention may be utilized with hydroxypropyl methylcellulose essentially as described in U.S. Pat. No. 4,916,138, incorporated herein by reference, or with a surfactant essentially as described in EPO patent publication No. 428,169, incorporated herein by reference.

Oral dosage forms may be prepared essentially as described by Hondo et al., 1987, Transplantation Proceedings XIX, Supp. 6:17-22, incorporated herein by reference. Dosage forms for external application may be prepared essentially as described in EPO patent publication No. 423,714, incorporated herein by reference. The active compound is included in the pharmaceutical composition in an amount sufficient to produce the desired effect upon the disease process or condition.

For the treatment of conditions and diseases caused by infection, immune system disorder (or to suppress immune function), or cancer, a compound of the invention may be administered orally, topically, parenterally, by inhalation spray, or rectally in dosage unit formulations containing conventional non-toxic pharmaceutically acceptable carriers, adjuvant, and vehicles. The term parenteral, as used herein, includes subcutaneous injections, and intravenous, intrathecal, intramuscular, and intrasternal injection or infusion techniques.

Dosage levels of the compounds are of the order from about 0.01 mg to about 100 mg per kilogram of body weight per day, preferably from about 0.1 mg to about 50 mg per kilogram of body weight per day. The dosage levels are useful in the treatment of the above-indicated conditions (from about 0.7 mg to about 3.5 mg per patient per day, assuming a 70 kg patient). In addition, the compounds of the present invention may be administered on an intermittent basis, i.e., at semi-weekly, weekly, semi-monthly, or monthly intervals.

The amount of active ingredient that may be combined with the carrier materials to produce a single dosage form will vary depending upon the host treated and the particular mode of administration. For example, a formulation intended for oral administration to humans may contain from 0.5 mg to 5 gm of active agent compounded with an appropriate and convenient amount of carrier material, which may vary from about 5 percent to about 95 percent of the total composition. Dosage unit forms will generally contain from about 0.5 mg to about 500 mg of active ingredient. For external administration, the compounds of the invention may be formulated within the range of, for example, 0.00001% to 60% by weight, preferably from 0.001% to 10% by weight, and most preferably from about 0.005% to 0.8% by weight.

It will be understood, however, that the specific dose level for any particlular patient will depend on a variety of factors. These factors include the activity of the specific compound employed; the age, body weight, general health, sex, and diet of the subject; the time and route of administration and the rate of excretion of the drug; whether a drug combination is employed in the treatment; and the severity of the particular disease or condition for which therapy is sought.

A detailed description of the invention having been provided above, the following examples are given for the purpose of illustrating the present invention and shall not be construed as being a limitation on the scope of the invention or claims.

### EXAMPLE 1

### Heterologous Expression of EpoK and EpoK Inhibitor Assays

This Example describes the construction of E. coli expression vectors for heterologous expression of the Sorangium cellulosum epoK gene. The E. coli produced EpoK enzyme can be used in assays to identify inhibitors for use in the methods of the invention. The epoK gene product was expressed in E. coli as a fusion protein with a polyhistidine tag (his tag). The fusion protein was purified and used to convert epothilone D to epothilone B. This assay can be readily adapted to identify EpoK inhibitors.

Plasmids were constructed to encode fusion proteins composed of six histidine residues fused to either the amino or carboxy terminus of EpoK. The following oligonucleotides were used to construct the plasmids:

| | |
|---|---|
| 55-101.a-1: | 5′-AAAAACATATGCACCACCACCACCACCACATGACACAGGAGCAAGCGAAT- |
| | CAGAGTGAG-3′, |
| 55-101.b: | 5′-AAAAAGGATCCTTAATCCAGCTTTGGAGGGCTT-3′, |
| 55-101.c: | 5′-AAAAACATATGACACAGGAGCAAGCGAAT-3′, and |
| 55-101.d: | 5′-AAAAAGGATCCTTAGTGGTGGTGGTGGTGGTGTCCAGCTTTGGAGGGCTTC- |
| | AAGATGAC-3′. |

The plasmid encoding the amino terminal his tag fusion protein, pKOS55-121, was constructed using primers 55-101.a-1 and 55-101.b, and the one encoding the carboxy terminal his tag, pKOS55-129, was constructed using primers 55-101.c and 55-101.d in PCR reactions containing pKOS35-83.5 as the template DNA. Plasmid pKOS35-83.5 contains the -5 kb NotI fragment comprising the epoK gene ligated into pBluescriptSKII+(Stratagene). The PCR products were cleaved with restriction enzymes BamHI and NdeI and ligated into the BamHI and NdeI sites of pET22b (Invitrogen). Both plasmids were sequenced to verify that no mutations were introduced during the PCR amplification. Protein gels were run as known in the art.

Purification of EpoK was performed as follows. Plasmids pKOS55-121 and pKOS55-129 were transformed into BL21(DE3) containing the groELS expressing plasmid pREP4-groELS (Caspers et al., 1994, Cellular and Molecular Biology 40(5):635-644). The strains were inoculated into 250 mL of M9 medium supplemented with 2 mM MgSO4, 1% glucose, 20 mg thiamine, 5 mg FeCl₂, 4 mg CaCl₂ and 50 mg levulinic acid. The cultures were grown to an OD₆₀₀ between 0.4 and 0.6, at which point IPTG was added to 1 mM, and the cultures were allowed to grow for an additional two hours. The cells were harvested and frozen at −80° C. The frozen cells were resuspended in 10 ml of buffer 1 (5 mM imidizole, 500 mM NaCl, and 45 mM Tris pH 7.6) and were lysed by sonicating three times for 15 seconds each on setting 8. The cellular debris was pelleted by centrifugation in an SS-34 rotor at 16,000 rpm for 30 minutes. The supernatant was removed and centrifuged again at 16,000 rpm for 30 minutes. The supernatant was loaded onto a 5 mL nickel column (Novagen), after which the column was washed with 50 mL of buffer 1 (Novagen). EpoK was eluted with a gradient from 5 mM to 1M imidizole. Fractions containing EpoK were pooled and dialyzed twice against 1 L of dialysis buffer (45 mM Tris pH7.6, 0.2 mM DTT, 0.1 mM EDTA, and 20% glycerol). Aliquots were frozen in liquid nitrogen and stored at −80° C. The protein preparations were greater than 90% pure.

The EpoK assay was performed as follows. Briefly, reactions consisted of 50 mM Tris (pH7.5), 21 gM spinach ferredoxin, 0.132 units of spinach ferredoxin: NADP⁺oxidoreductase, 0.8 units of glucose-6-phosphate dehydrogenase, 1.4 mM NADP, and 7.1 mM glucose-6-phosphate, 100 μM or 200 ,μM epothilone D (a generous gift of S. Danishefsky), and 1.7 ,μM amino terminal his tagged EpoK or 1.6 μM carboxy terminal his-tagged EpoK in a 100 μL volume. The reactions were incubated at 30° C. for 67 minutes and stopped by heating at 90° C. for 2 minutes. The insoluble material was removed by centrifugation, and 50 μL of the supernatant were analyzed by LC/MS. The reactions containing EpoK and epothilone D contained a compound absent in the control that displayed the same retention time, molecular weight, and mass fragmentation pattern as pure epothilone B. With an epothilone D concentration of 100 μM, the amino and the carboxy terminal his tagged EpoK were able to convert 82% and 58% to epothilone B, respectively. In the presence of 200 μM, conversion was 44% and 21%, respectively. These results demonstrate that EpoK can convert epothilone D to epothilone B.

To implement the protocol for identifying irreversible inhibitors of EpoK, one preferably first determines the Km of EpoK for epothilone D. Reaction mixtures (100 μl) are prepared as described for assay of EpoK, above, except the concentration of epothilone D is varied from 20 μM to 200 μM, and at each substrate concentration, initial velocities are determined. The Km is determined from a plot of initial velocity vs. epothilone D concentration. It may be necessary to adjust the range of substrate concentrations chosen for Km determination, such that the substrate concentration at 1/2 Vmax (i.e., Km) lies in the middle of the range. Once this determination is made, one next measures time dependent inhibition with a putative inhibitor compound. To do this, EpoK at a 10-fold higher concentration than that used in the enzymatic assay is pre-incubated in assay buffer with inhibitor, initially at a concentration of 1 mM. At various times, the pre-incubation mixture is diluted 10-fold into a reaction mixture containing epothilone D at a concentration of 10×Km. Enzyme activity vs. time is plotted, and irreversible inhibition is characterized by a time-dependent decrease in activity, compared to control with no added inhibitor.

With this methodology, one can test a wide variety of potential inhibitor compounds for inhibitory activity against EpoK. Compounds found to be irreversible inhibitors can be employed in the methods of the present invention.

### EXAMPLE 2

### Synthesis of EpoK Inhibitors

This example describes the synthesis of a number of preferred EpoK inhibitor compounds of the invention: 1-phenyl-3-butyn-1-yl-acetate, 1-phenylhexen-5-yn-3-yl acetate, 1-(3-pyridyl)-3-butyn-1-yl acetate 1-(3-pyridyl)hexen-5-yn-3-yl acetate, 1-(4-pyridyl)-3-butyn-1-yl acetate, 1-(4-pyridyl)hexen-5-yn-3-yl acetate, and 1-(2-methyl-4-thiazolyl)-2-methylhex-1-en-5-yn-3-yl acetate.

### A.

### Synthesis of 1-phenyl-3-butyn-1-ol

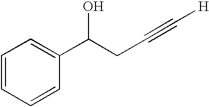

Propargyl bromide (1 mL, 80% in toluene) was added dropwise to a suspension of magnesium turnings (2.0 g) and zinc chloride (5 mL of a 1 M solution in ether) in dry tetrahydrofuran (10 mL). An exothermic reaction ensued, after which a mixture of propargyl bromide (10 mL, 80% in toluene) and benzaldehyde (5 mL) was added dropwise at such a rate so as to maintain a gentle reflux. After addition, the reaction was warmed to maintain reflux for 3 hours, then allowed to cool overnight. The resulting dark mixture was poured into dilute H₂SO₄ with stirring and diluted with ether. The pH of the aqueous phase was adjusted to pH 4, and the phases were separated. The organic phase was washed sequentially with 2 N HCl, saturated NaHCO₃, and brine, then dried over MgSO₄, filtered, and evaporated to a thick yellow oil. Distillation under vacuum yielded the product, a colorless oil. ¹H-NMR (CDCl₃, 400 MHz): δ7.4-7.2 (5H,m), 4.86 (1H,t,J=6.4 Hz), 2.63 (2H,dd,J=2.4, 6.4 Hz), 2.06 (1H,t,J=2.4 Hz). ¹³C-NMR (CDCl₃, 100 MHz): δ142.46, 128.56, 127.99, 125.75, 80.68, 72.33, 70.96, 29.43.

### B.

### Synthesis of 1-phenyl-3-butyn-1-yl acetate

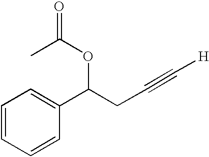

A mixture of 1-phenyl-3-butyn-1-ol (1.0 g), pyridine (1 mL) and acetic anhydride (2 mL in 5 mL of ether was cooled on ice, and 4-(dimethylamino)pyridine (100 mg) was added. After 1 hour, the mixture was diluted with ether and washed sequentially with 2 N HCl, saturated NaHCO₃, and brine, then dried over MgSO₄, filtered, and evaporated to an oil. Chromatography on silica gel (5:1 hexanes/ether) yielded 1.04 g of the pure product. ¹H-NMR (CDCl₃, 400 MHz): δ7.4-7.2 (5H,m), 5.89 (1H,t,J=6.8 Hz), 2.78 (1H,ddd,J=2.7, 7.0, 16.8 Hz), 2.70 (1H,ddd,J=2.7, 10.4, 16.8 Hz), 2.10 (3H,s), 1.97 (1H,t,J=2.4 Hz). ¹³C-NMR (CDCl₃, 100 MHz): δ169.97, 138.95, 128.46, 128.37, 126.51, 79.44, 73.49, 70.66, 26.46, 21.06, 21.08.

### C.

### Synthesis of 1-phenyl-1-hexen-5-yn-3-ol

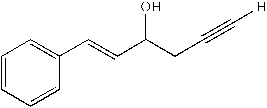

Propargyl bromide (1 mL, 80% in toluene) was added dropwise to a suspension of magnesium turnings (2.0 g) and zinc chloride (5 mL of a 1 M solution in ether) in dry tetrahydrofuran (10 mL). After the initial exothermic reaction, a mixture of propargyl bromide (10 mL, 80% in toluene) and cinnamaldehyde (5 mL) was added dropwise at such a rate so as to maintain a gentle reflux. After addition, the reaction was warmed to maintain reflux for 1 hour, then allowed to cool. The resulting dark mixture was poured into dilute H₂SO₄ with stirring and diluted with ether. The pH of the aqueous phase was adjusted to pH 4, and the phases were separated. The organic phase was washed sequentially with 2 N HCl, saturated NaHCO₃, and brine, then dried over MgSO₄, filtered, and evaporated. Silica gel chromatography (1:1 ether/hexane) yielded the product. ¹H-NMR (CDCl₃, 400 MHz): δ7.4-7.2 (5H,m), 6.65 (1H,d,J=16 Hz), 6.26 (1H,dd,J=6,16 Hz), 4.47 (1H,m), 2.58 (1H,ddd,J=2.7, 5.5, 16.8 Hz), 2.52 (1H,ddd,J=2.7, 6.4, 16.8 Hz), 2.24 (1H,br d,J=4 Hz), 2.08 (1H,t,J=2.7 Hz). ¹³C-NMR (CDCl₃, 100 MHz): δ136.36, 131.33, 130.01, 128.60, 127.89, 126.61, 80.25, 71.11, 70.71, 27.74.

### D.

### Synthesis of 1-phenyl-1-hexen-5-yn-3-yl acetate

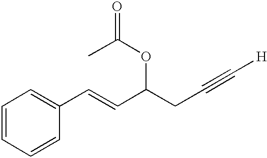

A mixture of 1-phenyl-1-hexen-5-yn-3-ol (1.0 g), pyridine (1 mL) and acetic anhydride (2 mL in 5 mL of ether is cooled on ice, and 4-(dimethylamino)pyridine (100 mg) is added. After 1 hour, the mixture is diluted with ether and washed sequentially with 2 N HCl, saturated NaHCO₃, and brine, then dried over MgSO₄, filtered, and evaporated. Chromatography on silica gel (5:1 hexanes/ether) yields the pure product. ¹H-NMR (CDCl₃, 400 MHz): δ7.4-7.2 (5H,m), 6.65 (1H,d,J=16 Hz), 6.23 (1H,dd,J=7,16 Hz), 5.53 (1H,ddd,J=1,3,6), 2.62 (2H,ddJ=3,6), 2.10 (3H,s), 2.03 (1H,t,J=2.8 Hz). ¹³C-NMR (CDCl₃, 100 MHz): δ170.10, 135.98, 133.57,128.60, 128.18, 126.73, 125.66, 79.28, 72.20, 70.77, 24.90, 21.21.

### E.

### Synthesis of 1-(3-pyridyl)-3-butyn-1-ol

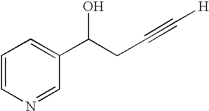

Propargyl bromide (1 mL, 80% in toluene) is added dropwise to a suspension of magnesium turnings (2.0 g) and zinc chloride (5 mL of a 1 M solution in ether) in dry tetrahydrofuran (10 mL). An exothermic reaction ensues, after which a mixture of propargyl bromide (10 mL, 80% in toluene) and 3-pyridinecarboxaldehyde (5 mL) is added dropwise at such a rate so as to maintain a gentle reflux. After addition, the reaction is warmed to maintain reflux for 3 hours, then allowed to cool overnight. The resulting dark mixture is poured into dilute H₂SO₄ with stirring and diluted with ether. The pH of the aqueous phase is adjusted to pH 4, and the phases are separated. The organic phase is washed sequentially with 2 N HCl, saturated NaHCO₃, and brine, then dried over MgSO₄, filtered, and evaporated. Distillation under vacuum yields the product.

### F.

### Synthesis of 1-(3-pyridyl)-3-butyn-1-yl acetate

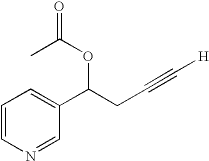

A mixture of 1-(3-pyridyl)-3-butyn-1-ol (1.0 g), pyridine (1 mL) and acetic anhydride (2 mL in 5 mL of ether is cooled on ice, and 4-(dimethylarnino)pyridine (100 mg) is added. After 1 hour, the mixture is diluted with ether and washed sequentially with 2 N HCl, saturated NaHCO₃, and brine, then dried over MgSO₄, filtered, and evaporated. Chromatography on silica gel yields the pure product.

### G.

### Synthesis of 1-(3-pyridyl)hexen-5-yn-3-ol

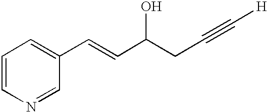

Propargyl bromide (1 mL, 80% in toluene) is added dropwise to a suspension of magnesium turnings (2.0 g) and zinc chloride (5 mL of a 1 M solution in ether) in dry tetrahydrofuran (10 mL). An exothermic reaction ensues, after which a mixture of propargyl bromide (10 mL, 80% in toluene) and 3-(3-pyridyl)propenal (5 mL) is added dropwise at such a rate so as to maintain a gentle reflux. After addition, the reaction is warmed to maintain reflux for 3 hours, then allowed to cool overnight. The resulting dark mixture is poured into dilute H₂SO₄ with stirring and diluted with ether. The pH of the aqueous phase is adjusted to pH 4, and the phases are separated. The organic phase is washed sequentially with 2 N HCl, saturated NaHCO₃, and brine, then dried over MgSO₄, filtered, and evaporated. Distillation under vacuum yields the product.

### H.

### Synthesis of 1-(3-pyridyl)hexen-5-yn-3-yl acetate

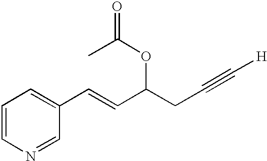

A mixture of 1-(3-pyridyl)hexen-5-yn-3-ol (1.0 g), pyridine (1 mL) and acetic anhydride (2 mL in 5 mL of ether is cooled on ice, and 4-(dimethylamino~pyridine (100 mg) is added. After 1 hour, the mixture is diluted with ether and washed sequentially with 2 N HCl, saturated NaHCO₃, and brine, then dried over MgSO₄, filtered, and evaporated. Chromatography on silica gel yields the pure product.

### I.

### Synthesis of 1-(4-pyridyl)-3-butyn-1-ol

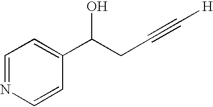

Propargyl bromide (1 mL, 80% in toluene) is added dropwise to a suspension of magnesium turnings (2.0 g) and zinc chloride (5 mL of a 1 M solution in ether) in dry tetrahydrofuran (10 mL). An exothermic reaction ensues, after which a mixture of propargyl bromide (10 mL, 80% in toluene) and 4-pyridinecarboxaldehyde (5 mL) is added dropwise at such a rate so as to maintain a gentle reflux. After addition, the reaction is warmed to maintain reflux for 3 hours, then allowed to cool overnight. The resulting dark mixture is poured into dilute H₂SO₄ with stirring and diluted with ether. The pH of the aqueous phase is adjusted to pH 4, and the phases are separated.

The organic phase is washed sequentially with 2 N HCl, saturated NaHCO₃, and brine, then dried over MgSO₄, filtered, and evaporated. Distillation under vacuum yields the product.

### J.

### Synthesis of 1-(4-pyridyl)-3-butyn-1-yl acetate

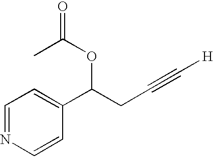

A mixture of 1-(4-pyridyl)-3-butyn-1-ol (1.0 g), pyridine (1 mL) and acetic anhydride (2 mL in 5 mL of ether is cooled on ice, and 4-(dimethylamino)pyridine (100 mg) is added. After 1 hour, the mixture is diluted with ether and washed sequentially with 2 N HCl, saturated NaHCO₃, and brine, then dried over MgSO₄, filtered, and evaporated. Chromatography on silica gel yields the pure product.

### K.

### Synthesis of 1-(4-pyridyl)hexen-5-yn-3-ol

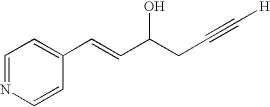

Propargyl bromide (1 mL, 80% in toluene) is added dropwise to a suspension of magnesium turnings (2.0 g) and zinc chloride (5 mL of a 1 M solution in ether) in dry tetrahydrofuran (10 mL). An exothermic reaction ensues, after which a mixture of propargyl bromide (10 mL, 80% in toluene) and 3-(4-pyridyl)propenal (5 mL) is added dropwise at such a rate so as to maintain a gentle reflux. After addition, the reaction is warmed to maintain reflux for 3 hours, then allowed to cool overnight. The resulting dark mixture is poured into dilute H₂SO₄ with stirring and diluted with ether. The pH of the aqueous phase is adjusted to pH 4, and the phases are separated. The organic phase is washed sequentially with 2 N HCl, saturated NaHCO₃, and brine, then dried over MgSO₄, filtered, and evaporated. Distillation under vacuum yields the product.

### L.

### Synthesis of 1-(4-pyridyl)hexen-5-yn-3-yl acetate

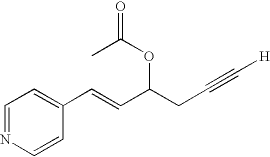

A mixture of 1-(4-pyridyl)hexen-5-yn-3-ol (1.0 g), pyridine (1 mL) and acetic anhydride (2 mL in 5 mL of ether is cooled on ice, and 4-(dimethylamino)pyridine (100 mg) is added. After 1 hour, the mixture is diluted with ether and washed sequentially with 2 N HCl, saturated NaHCO₃, and brine, then dried over MgSO₄, filtered, and evaporated. Chromatography on silica gel yields the pure product.

### M.

### Synthesis of 1-(2-methyl-4-thiazolyl)-2-methylhex-1-en-5-yn-3-yl acetate

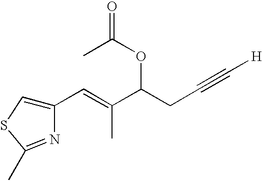

This compound is prepared according to literature procedures (Bin Zhu and James S. Panek, Tetrahedron Letters (2000) 41(12), 1863-1866).

### EXAMPLE 3

### Producing Epothilones and Epothilone Derivatives in Sorangium cellulosum

This example describes a fermentation protocol for epothilone producing Sorangium cellulosum strains. A fresh plate of Sorangium cellulosum strain SMP44 host cells (dispersed) is prepared on S42 medium (other strains, such as So ce 90 (DSM 6773), can also be used). S42 medium contains tryptone, 0.5 g/L; MgSO₄, 1.5 g/L; HEPES, 12 g/L; agar, 12 g/L, with deionized water. The pH of S42 medium is set to 7.4 with KOH. To prepare S42 medium, after autoclaving at 121° C. for at least 30 minutes, add the following ingredients (per liter): CaCl₂, 1 g; K₂HPO₄, 0.06 g; Fe Citrate, 0.008 g; Glucose, 3.5 g; Ammonium sulfate, 0.5 g; Spent liquid medium, 35 mL; and 200 micrograms/mL of kanamycin is added to prevent contamination. Incubate the culture at 32° C. for 4-7 days, or until orange sorangia appear on the surface.

To prepare a seed culture for inoculating agar plates/bioreactor, the following protocol is followed. A patch of Sorangium cells is scraped from the agar (about 5 mm²) and transferred to a 250 ml baffle flask with 38 mm silicone foam closures containing 50 ml of Soymeal Medium containing potato starch, 8 g; defatted soybean meal, 2 g; yeast extract, 2 g; Iron (III) sodium salt EDTA, 0.008 g; MgSO₄.7H₂O, 1 g; CaCl₂.2H₂O, 1 g; glucose, 2 g; HEPES buffer, 11.5 g. Use deionized water, and adjust pH to 7.4 with 10% KOH. Add 2-3 drops of antifoam B to prevent foaming. Incubate the culture in a shaker for 4-5 days at 30° C. and 250 RPM. The culture should appear an orange color. This seed culture can be subcultured repeatedly for scale-up to inoculate in the desired volume of production medium.

The same preparation can be used with Medium 1 containing (per liter) CaCl₂.2H₂O, 1 g; yeast extract, 2 g; Soytone, 2 g; FeEDTA, 0.008 g; Mg SO₄.7H₂O, 1 g; HEPES, 11.5 g. Adjust pH to 7.4 with 10% KOH, and autoclave at 121° C. for 30 minutes. Add 8 ml of 40% glucose after sterilization. Instead of a baffle flask, use a 250 ml coiled spring flask with a foil cover. Include 2-3 drops of antifoam B, and incubate in a shaker for 7 days at 37° C. and 250 RPM. Subculture the entire 50 mL into 500 mL of fresh medium in a baffled narrow necked Fernbach flask with a 38 mm silicone foam closure. Include 0.5 ml of antifoam to the culture. Incubate under the same conditions for 2-3 days. Use at least a 10% inoculum for a bioreactor fermentation.

To culture on solid media, the following protocol is used. Prepare agar plates containing (per liter of CNS medium) KNO₃, 0.5 g; Na₂HPO₄, 0.25 g; MgSO₄.7H₂O, 1 g; FeCl₂, 0.01 g; HEPES, 2.4 g; Agar, 15 g; and sterile Whatman filter paper. While the agar is not completely solidified, place a sterile disk of filter paper on the surface. When the plate is dry, add just enough of the seed culture to coat the surface evenly (about 1 mL). Spread evenly with a sterile loop or an applicator, and place in a 32° C. incubator for 7 days. Harvest plates.

For production in a 5 L bioreactor, the following protocol is used. The fermentation can be conducted in a B. Braun Biostat MD-1 5L bioreactor. Prepare 4 L of production medium (same as the sovmeal medium for the seed culture without HEPES buffer). Add 2% (volume to volume) XAD-16 absorption resin, unwashed and untreated, e.g. add 1 mL of XAD per 50 mL of production medium. Use 2.5 N H₂SO₄ for the acid bottle, 10% KOH for the base bottle, and 50% antifoam B for the antifoam bottle. For the sample port, be sure that the tubing that will come into contact with the culture broth has a small opening to allow the XAD to pass through into the vial for collecting daily samples. Stir the mnixture completely before autoclaving to evenly distribute the components. Calibrate the pH probe and test dissolved oxygen probe to ensure proper functioning. Use a small antifoam probe, ˜3 inches in length. For the bottles, use tubing that can be sterile welded, but use silicone tubing for the sample port. All fittings should be secure, and the tubes clamped shut with C-clamps. (do not clamp the tubing to the exhaust condenser). Attach 0.2 μm filter disks to any open tubing in contact with the air. Use larger ACRO 50 filter disks for larger tubing, such as the exhaust condenser and the air inlet tubing. Prepare a sterile empty bottle for the inoculum. Autoclave at 121° C. with a sterilization time of 90 minutes. Once the reactor has been taken out of the autoclave, connect the tubing to the acid, base, and antifoam bottles through their respective pump heads. Release the clamps to these bottles, making sure the tubing has not been welded shut. Attach the temperature probe to the control unit. Allow the reactor to cool, while sparging with air through the air inlet at a low air flow rate.

After ensuring the pumps are working and there is no problem with flow rate or clogging, connect the hoses from the water bath to the water jacket and to the exhaust condenser. Make sure the water jacket is nearly full. Set the temperature to 32° C. Connect pH, D.O., and antifoam probes to the main control unit. Test the antifoam probe for proper functioning. Adjust the set point of the culture to 7.4. Set the agitation to 400 RPM. Calibrate the dissolved oxygen (D.O.) probe using air and nitrogen gas. Adjust the airflow using the rate at which the fermentation will operate, e.g. 1 LPM (liter per minute). To control the D.O. level, adjust the parameters under the cascade setting so that agitation will compensate for lower levels of air to maintain a D.O. value of 50%. Set the minimum and maximum agitation to 400 and 1000 RPM respectively, based on the settings of the control unit. Adjust the settings, if necessary.

Check the seed culture for any contamination before inoculating the fermenter. The Sorangium cellulosum cells are rod shaped like a pill, with 2 large distinct circular vacuoles at opposite ends of the cell. Length is approximately 5 times that of the width of the cell. Use a 10% inoculum (minimum) volume, e.g. 400 mL into 4 L of production medium. Take an initial sample from the vessel and check against the bench pH. If the difference between the fermenter pH and the bench pH is off by ≧0.1 units, do a 1 point recalibration. Adjust the deadband to 0.1. Take daily 25 mL samples noting fermenter pH, bench pH, temperature, D.O., airflow, agitation, acid, base, and antifoam levels. Adjust pH if necessary. Allow the fermenter to run for seven days before harvesting.

The liquid cultures are extracted three times with equal volumes of ethyl acetate, the organic extracts combined and evaporated, and the residue dissolved in acetonitrile for LC/MS analysis. The agar plate media is chopped and extracted twice with equal volumes of acetone, and the acetone extracts are combined and evaporated to an aqueous slurry, which is extracted three times with equal volumes of ethyl acetate. The organic extracts are combined and evaporated, and the residue dissolved in acetonitrile for LC/MS analysis.

Production of epothilones can be assessed using LC-mass spectrometry. The output flow from the Uw detector of an analytical HPLC is split equally between a Perkin-Elmer/Sciex API100LC mass spectrometer and an Alltech 500 evaporative light scattering detector. Samples are injected onto a 4.6×150 mm reversed phase HPLC column (MetaChem 5 m ODS-3 Inertsil) equilibrated in water with a flow rate of 1.0 mL/min. Lw detection is set at 250 nm. Sample components are separated using H₂O for 1 minute, then a linear gradient from 0 to 100% acetonitrile over 10 minutes. Under these conditions, epothilone A elutes at 10.2 minutes and epothilone B elutes at 10.5 minutes. The identity of these compounds can be confirmed by the mass spectra obtained using an atmospheric chemical ionization source with orifice and ring voltages set at 75 V and 300 V, respectively, and a mass resolution of 0.1 amu.

To practice the method of the invention, the fermentation protocol described above can be followed, with one or more of the inhibitors of the invention added to the fermentation media to achieve a final concentration in the media in the range of 1 nM to 1 M, preferably in the range of 1 μM to 100 mM, most preferably in the range of 10 μM to 10 mM. The inhibitor can be added to the fermentation as a bolus or in aliquots over time; typically, the inhibitor will be added prior to the start of production of the epothilones.

### EXAMPLE 4

### Production of Epothilone C and D with EpoK Inhibitor Metyrapone

This example describes a protocol for producing epothilones C and D by inhibition of epoK with metyrapone (2-methyl-1,2-di-3-pyridyl-1-propanone) in the strain Sorangium cellulosum So ce 90. So ce 90 can be obtained from the German Collection of Microorganisms under accession number DSM 6773.

To prepare a seed culture, transfer cells from the DSM 6773 ampoule into a 250 mL baffled Erleneyer flask with 38 mm silicone foam closures containing 50 mL of sterile soybean meal. Soybean meal contains potato starch (Product No. S-2004, Sigma), 8 g/L; defatted soybean meal (Type 4890, Archer Daniels Midland), 2 g/L; yeast extract (Product No. BP 1422-500, Fisher Biotech), 2g/L; Iron (III) sodium salt EDTA (Product No. EDFS, Sigma), 0.008 g/L; MgSO₄.7H₂0 (Product No. M-5921, Sigma), 1 g/L; CaCl₂.2H₂0 (Product No. C-3991, Sigma), 1 g/L; glucose (Product No. G-5400, Sigma), 2g/L; HEPES buffer (Product No. H-3375, Sigma), 11.5 g/L. Use deionized water, and adjust pH to 7.4 with 10% KOH. Sterilize the flasks by autoclaving at 121° C. for at least 30 minutes. After autoclaving, add 2-3 drops of antifoam B to prevent foaming. Incubate the culture at 32° C. in a 250 RPM shaker for 4 days. After 4 days, the culture should appear an orange color. Production cultures were prepared by sterilizing 50 mL of sterile soybean meal medium and 1 g of Amberlite XAD-16 absorption resin in a 250 mL baffled Erlenmeyer flask. Metyrapone (Product No. 856525, Sigma) is prepared by reconstituting to a final concentration of 2.5 M with 50:50 (v/v) DMSO:H2O. Metyrapone was added to a final concentration of 5 mM and 10 mM in each flask. 5 mL of the seed culture was inoculated to each flask and incubated at 30° C. in a shaker at 250 RPM for 7 days.

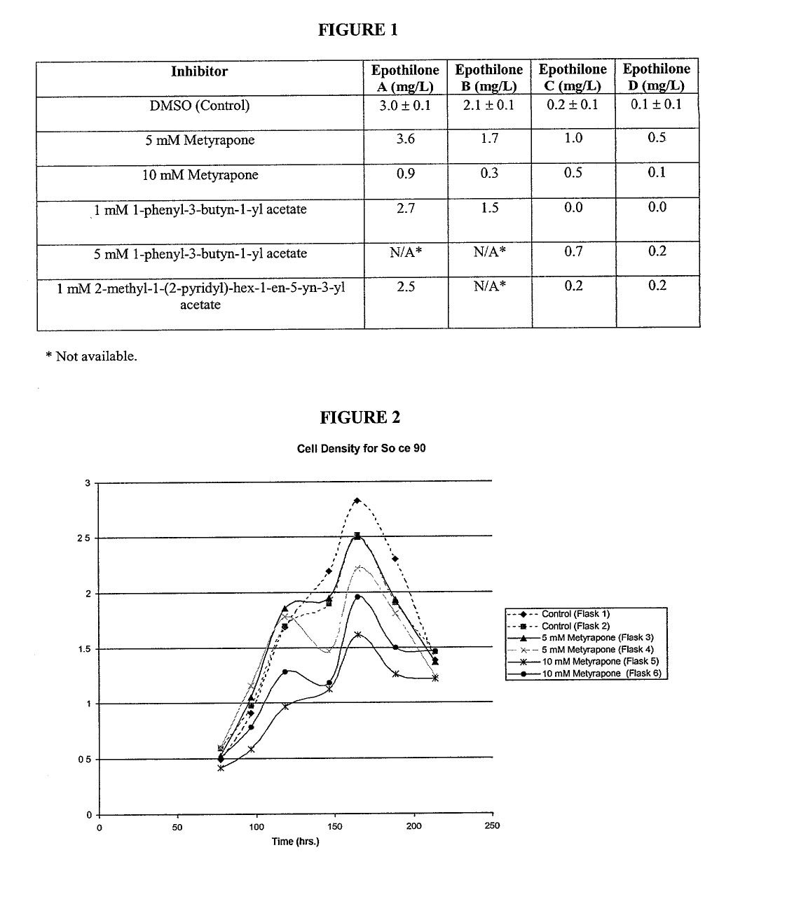

To extract epothilone C and/or D, transfer the culture into a 50 mL centrifuge tube (Product no. 21008-178, VWR Scientific Products). Decant the excess medium in the centrifuge tube without pouring off any of the resin. Wash the XAD-16 resin with 25 mL of H₂O and allow the XAD-16 resin to settle by gravity in the tube. Carefully decant the H₂O, and add 20 mL of 100% methanol to the tube. Place the centrifuge tube on a shaker at 175 RPM for 20-30 minutes to extract the epothilone products from the resin. Allow the XAD-16 pellets to settle, and transfer 2 mL using a pipette to a HPLC tube (Product nos. C4010-13, C4010-60A, National Scientific Company). Quantitation of epothilones C and D was performed by HPLC analysis with UV-DAD detection at 250 nm. 50 uL of the methanol extract was injected across a 4.6×10 mm guard column (Inertsil, C18 OD 53, 5um) and a 4.6 x 150 mm guard column (Inertisil, C18 OD 53, 5 um). The assay method was isocratic, eluting with 60% acetonitrile and 40% water for 18 minutes at a flow rate of 1 ml/min. Under these conditions, epothilone C was detected at 10.3 minutes and epothilone D was detected at 13.0 minutes. The results showing the highest production of epothilones C and D, and the lowest production of epothilones A and B, in the presence of metyrapone and various other inhibitors is shown in FIG. 1. As shown in FIG. 1, the So ce 90 strain produced 3.6 mg/L and 1.7 mg/L of epothilones A and B, respectively, and 1.0 mg/L and 0.5 mg/L of epothilones C and D, respectively. In the presence of 10 mM metyrapone, the So ce 90 strain produced 0.9 mg/L and 0.3 mg/L of epothilones A and B, respectively, and 0.5 mg/L and 0.1 mg/L of epothilones C and D, respectively. FIG. 2 shows that metyrapone at the concentrations of 5 mM and 10 mM does not detrimentally inhibit the growth of the Sorangium cellulosum strain So ce 90.

### EXAMPLE 5

### Mutagenesis of Sorangum cellulosum strain So ce 90

This example describes a protocol for obtaining mutant strains of Sorangium cellulosum that contain a mutationally inactivated epoK gene. Sorangium cellulosum So ce 90 is obtained from the German Collection of Microorganisms under accession number DSM 6773.

The cells of the DSM 6773 ampoule are transferred to 10 mL of G52 medium in a 50 mL Erlenmeyer flask and incubated for 6 days in an agitator at 30° C. and 180 rpm. G52 medium contains 2 g/L yeast extract, low in salt (Springer, Maison Alfort, France); 1 g/L MgSO₄ (7H₂O); 1 g/L CaCl₂(2H₂O); 2 g/L soya meal defatted (Mucedola S.r.l., Settimo Milan, Italy); 8 g/L potato starch Noredux (Blattmann, Wadenswil, Switzerland); 2 g/L glucose anhydrous; 1 mL/L Fe-EDTA 8g/L (Product No. 03625, Fluka Chemie AG, CH); the pH is adjusted to 7.4 with KOH; and the medium is sterilized at 120° C. for 20 minutes. About 5 mL of this culture are transferred to 50 mL of G52 medium (in a 200 mL Erlenmeyer flask) and incubated at 180 rpm for 3 days in an agitator at 30° C.

Portions of 0.1 mL of the above culture are plated out onto several Petri dishes containing agar medium S42 (Jaoua et al., 1992, Plasmid 28: 157 -165). The plates are then each exposed to UV light (maximum radiation range of 250 -300 nm) for 90 to 120 seconds at 500 μ watt per cm². The plates are then incubated for 7 -9 days at 30° C., until individual colonies of 1-2 mm are obtained. The cells of 100 -150 colonies are then each plated out from an individual colony by means of plastic loop in sectors on Petri dishes containing S42 agar (4 sectors per plate) and incubated for 7 days at 30° C. The cells that have grown on an area of ca. 1 cm² agar are transferred by a plastic loop to 10 mL of G52 medium in a 50 mL Erlenmeyer flask and incubated for 7 daysat 180 rpm in an agitator at 30° C. About 5 mL of this culture are transferred to 50 mL of G52 medium (in a 200 mL Erlenmeyer flask) and incubated at 180 rpm for 3 days in an agitator at 30° C. About 10 mL of this culture are transferred to 50 mL of 23B3 medium and incubated for 7 days at 180 rpm in an agitator at 30° C. 23B3 medium contains 2 g/L glucose; 20 g/L potato starch Noredux; 16 g/L soya meal defatted; 8 g/L Fe-EDTA; 5 g/L HEPES (Fluka, Buchs, Switzerland); 2% v/v polystyrene resin XAD16 (Rohm and Haas); deionized water; the pH is adjusted to 7.8 with NaOH; and the medium is sterilized at 120° C. for 20 minutes.

To determine the amounts of epothilones A, B, C, and D formed in this culture, the following procedure is used. The 50 mL culture solution is filtered through a nylon sieve (150 μm pore size), and the polystyrene resin Amberlite XAD16 retained on the sieve is rinsed with a little water and subsequently added together with the filter to a 50 mL centrifuge tube (Falcon Labware, Becton Dickinson AG Immengasse 7,4056 Basle). 10 mL of isopropanol (>99%) are added to the tube with the filter. Afterwards, the well sealed tube is shaken for one hour at 180 rpm in order to dissolve the epothilones, which are bonded to the resin, in the isopropoanol. Subsequently, 1.5 mL of the liquid is centrifuged, and ca. 0.8 mL of the supernatant is added using a pipette to a HPLC tube. The HPLC analysis of these samples is effected as described below. The HPLC analysis determines which culture contains the highest content of epothilones C and D with the lowest content of epothilones A and B. From the above-described sector plate of the corresponding colony (the plates having been stored at 4° C. in the interim), cells from ca. 1 cm² of agar area are transferred by a platic loop to 10 mL of G52 medium in a 50 mL Erlenrmeyer flask and are incubated for 7 days at 180 rpm in an agitator at 30° C. About 5 mL of this culture are transferred to 50 mL of G52 medium (in a 200 mL Erlenmeyer flask) and incubated at 180 rpm for 3 days in an agitator at 30° C.

While this first round of mutagenesis should produce an epoK mutant as desired, further rounds of mutagenesis and screening can be employed. For example, one could identify mutants that produce more of epothilones C and D or more of one epothilone, such as epothilone D, than another. For second, third and subsequent rounds of mutagenesis, the procedure is exactly the same as described above for the first round of mutagenesis, with the selected culture of the best colony from the prior mutagenesis step used in the subsequent step.

HPLC sample analysis is performed as follows. About 50 mL samples are mixed with 2 mL of polystyrene resin Amberlite XAD16 (Rohm & Haas, Frankfurt, Germany) and shaken at 180 rpm for one hour at 30° C. The resin is subsequently filtered using a 150 μm nylon sieve, washed with a little water and then added together with the filter to a 15 mL Nunc tube. The product is eluted from the resin as follows. About 10 mL of isopropanol (>99%) are added to the tube with the filter and the resin. Afterwards, the sealed tube is shaken for 30 minutes at room temperature on a Rota-Mixer (Labinco BV, Netherlands). Then, 2 mL of the liquid are centrifuged off and the supernatant is added using a pipette to the HPLC tubes. The HPLC columns are a Waters-Symetry C18, 100×4 mm, 3.5 μm WAT066220 and preliminary column 3.9×20 mm WAT054225. The solvents are A: 0.02% phosphoric acid; and B: acetonitrile (HPLC-quality). The gradient is 41% B from 0 to 7 minutes, 100(% B from 7.2 to 7.8 minutes, and 41% B from 8 to 12 minutes. The oven temperature is 30° C. The detection is 250 nm, UV-DAD detection. The injection volume is 10 μL. The retention times for epothilone A and B are 4.30 and 5.38 minutes, respectively.

Alternatively, analysis of the cultures can be carried out as described below. For analysis of cultures grown in the presence of XAD-16, the grown culture is centrifuged at low speed to pellet the XAD-16, and the supernatant is discarded. The XAD-16 is suspended in 1 mL of water and recentrifuged, with the supernatant again being discarded. The XAD-16 is allowed to air dry overnight. An equal volume of acetonitrile is added and the suspension is agitated gently for 1 hour, then centrifuged to pellet solids. The supernatant is collected and used for analysis. An aliquot of this extract (typically 20 -50 uL) is injected directly into the APCI source of a PE/Sciex API-100LC mass spectrometer, using acetonitrile at a flow rate of 0.3 mL/min. The spectrometer is set to collect ion current over a m/z range of 450-550 amu, with a mass resolution of 0.1 amu, using the multi-channel analysis mode. Data is accumulated for 2 minutes before injection of the next sample. The presence of epothilones A, B, C, and D is detected by the ion current at m/z=494.7, 508.7, 478.7, and 492.7, respectively.

For analysis of cultures grown without XAD-16, the grown culture is centrifuged at high speed to pellet cells. The supernatant is passed through a C18-solid phase extraction cartridge, which is then washed with water. Organics are eluted from the cartridge by rinsing with acetonitrile. An aliquot of this organic extract (typically 20 -50 uL) is injected directly into the APCI source of a PE/Sciex API-100LC mass spectrometer, using acetonitrile at a flow rate of 0.3 mL/min. The spectrometer is set to collect ion current over a m/z range of 450-550 amu, with a mass resolution of 0.1 amu, using the multi-channel analysis mode. Data is accumulated for 2 minutes before injection of the next sample. The presence of epothilones A, B, C, and D is detected by the ion current at m/z=494.7, 508.7, 478.7, and 492.7, respectively.

What is claimed is:

1. A method for producing a desoxyepothilone, which comprises fermentation of an epothilone producing microorganism in the presence of an inhibitor of an epothilone epoxidase.

2. The method of claim 1, wherein said desoxyepothilone is epothilone D.

3. The method of claim 1, wherein said desoxyepothilone is epothilone C.

4. The method of claim 1, wherein said desoxyepothilone is a mixture of epothilone C and epothilone D.

5. The method of claim 1, wherein said microorganism is Sorangium cellulosum.

6. The method of claim 1, wherein said inhibitor is 2-methyl-1,2-di-3-pyridyl-1-propanone.

7. The method of claim 1, wherein said inhibitor is selected from the group consisting of ketoconazole, itraconazole, miconazole, furafylline, sulfaphenazole, proadifen, and debrisoquin.

8. The method of claim 1, wherein said inhibitor is a member of the class of acetylenic mechanism-based irreversible inhibitors.

9. The method of claim 8, wherein said inhibitor is 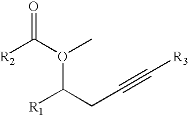
wherein R₁ is aryl, heterocycle, aryl—CH═CR₄—, or heterocycle-CH═CR₄; R₂ is lower alkyl, preferably C₁₋₃ alkyl; R₃ is H or is lower alkyl, preferably methyl, or ethyl; and R₄ is H or is lower alkyl, preferably methyl.

10. The method of claim 9, wherein said inhibitor is selected from the group consisting of 1-phenyl-3-butyn-1-yl-acetate, 1-phenylhexen-5-yn-3-yl acetate, 1-(3-pyridyl)-3-butyn-1-yl acetate 1-(3-pyridyl)hexen-5-yn-3-yl acetate, 1-(4-pyridyl)-3-butyn-1-yl acetate, and 1-(4-pyridyl)hexen-5-yn-3-yl acetate.

11. The method of claim 1, wherein said microorganism is Sorangium cellulosum, and said inhibitor is selected from the group consisting of 1-phenyl-3-butyn-1-yl-acetate, 1-phenylhexen-5-yn-3-yl acetate, 1-(3-pyridyl)-3-butyn-1-yl acetate 1-(3-pyridyl)hexen-5-yn-3-yl acetate, 1-(4-pyridyl)-3-butyn-1-yl acetate, and 1-(4-pyridyl)hexen-5-yn-3-yl acetate.

12. A recombinant Sorangium cellulosum host cell comprising an epoK gene that has been inactivated by mutation that produces epothilone C or epothilone D or both.

13. The host cell of claim 12 that produces more epothilone C and epothilone D than epothilone A and epothilone B.

14. The host cell of claim 12 that does not produce epothilone A or epothilone B.

15. The host cell of claim 12 that produces epothilone D but not epothilone C.

16. The host cell of claim 12 that produces epothilone C but not epothilone D.
